(19) United States
(12) Patent Application Publication
(10) Pub. No.: US 2024/0173472 A1
(43) Pub. Date: May 30, 2024

(54) DEVICES FOR INSERTING TRANSCUTANEOUS CANNULAS FOR PATCH PUMPS

(71) Applicant: Tandem Diabetes Care Switzerland Sàrl, St-Sulpice (CH)

(72) Inventors: Vincent VAUCHER, Sonceboz (CH); Pierre FRIDEZ, Froideville (CH)

(73) Assignee: Tandem Diabetes Care Switzerland Sàrl, St-Sulpice (CH)

(21) Appl. No.: 18/516,538

(22) Filed: Nov. 21, 2023

Related U.S. Application Data
(60) Provisional application No. 63/385,567, filed on Nov. 30, 2022.

(51) Int. Cl. A61M 5/142

(52) U.S. Cl. CPC A61M 5/14248; A61M 2005/1581

(57) ABSTRACT

Devices, systems, and methods are provided herein for assisting with transcutaneously implanting a cannula used for delivering medication (e.g., insulin). The implanted cannula may be used with a patch pump adhered to a wearer's body. An enhanced applicator needle is provided to cause the cannula transition from a straight configuration to a curved configuration during deployment within the skin. The applicator needle has a structural modification to cause the needle, and thereby cause the cannula having the needle positioned therein, to curve within the skin, for example, a special cut pattern at the needle tip and/or a bend at the distal region of the needle. Once transcutaneously implanted in the curved manner, the needle is fully withdrawn from the cannula so that a patch pump can be fluidly coupled to the curved cannula for transcutaneously delivering medication to a target infusion area beneath the wearer's outer skin layer.

### CROSS-REFERENCE TO RELATED APPLICATIONS

This application claims priority to U.S. Provisional Patent Application No. 63/385,567, filed Nov. 30, 2022, the entire contents of which are incorporated herein by reference.

### TECHNICAL FIELD

The technology relates to apparatus and methods for inserting devices into a patient, such as devices for inserting transcutaneous cannulas for a pump, e.g., wearable insulin pumps having a patch-style form factor for adhesion to a body surface.

### BACKGROUND

Wearable insulin pumps are known for providing a Type I Diabetes Mellitus patient with small doses of short acting insulin continuously (basal rate). The device also is used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

WO 02/20073 describes an ambulatory patch pump for delivering insulin to manage diabetes. The pump is part of a system that includes the fluid delivery device, a separate, remote control device, and accessories for transcutaneous delivery of fluid medications.

U.S. Pat. No. 7,879,026 describes an infusion pump that is designed to be wearable, e.g., on a user's belt, and is coupled to an infusion cannula that extends through and is fixed to a user's skin using an adhesive patch. The infusion pump may include an accelerometer or other motion sensor to detect the user's activity level, the output of which may be used to automatically adjust a rate of insulin infusion to the user based at least in part on a detected activity level of the user.

U.S. Pat. No. 9,735,893 describes a patch system for in-situ therapeutic treatment wherein a plurality of biological parameter monitoring devices may be disposed on separate stretchable patches designed to adhere to a user's skin. The monitoring devices communicate with each other, and other therapeutic devices, via short-range wireless, such as Bluetooth. The patent describes that patch-based monitoring devices may be configured to communicate to a belt-worn insulin pump, and that one patch-based monitoring device may include pulse oximetry electronics for measuring blood volume. The patent does not describe a patch-based insulin pump and requires intercommunication between its various components, providing a potential failure mode.

U.S. Pat. No. 11,806,502, the entire contents of which are incorporated herein by reference, assigned to the assignee of the instant application, describes a self-contained patch pump having a motor-actuated syringe together with a microdosing pump chamber.

U.S. Pat. No. 11,813,428, the entire contents of which are incorporated herein by reference, assigned to the assignee of the instant application, describes a drug delivery device comprising a pumping system and a liquid reservoir fluidly connected to a delivery system outlet. The liquid reservoir has an elastic plunger sealingly slidable within a container wall of the liquid reservoir for expelling liquid out of the reservoir.

U.S. Pat. No. 11,529,460, the entire contents of which are incorporated herein by reference, assigned to the assignee of the instant application, describes systems and methods for delivering medication such as insulin that are user-friendly, environmentally-friendly, lower cost, discreet, less prone to errors, and/or that deliver precise, repeatable doses of medication.

In patch pumps, it is often needed to insert a transcutaneous cannula into the skin for delivering medication transcutaneously from an external fluid reservoir associated with the patch pump that is in fluid communication with the cannula. Thus, the cannula needs to be inserted into the skin before delivering the medication. U.S. Patent App. Pub. No. 2019/0133505 to Jäger describes a medical device with a pre-bended insertion cannula. That cannula, however, is pre-bent before insertion into the patient's skin. U.S. Patent App. Pub. No. 2009/0069750 to Schraga and U.S. Patent App. Pub. No. 2014/0288399 to Regittnig describe straight needles for inserting a cannula in a perpendicular or angled manner. U.S. Patent App. Pub. No. 2013/0245555 to Dirac describes a straight needle for inserting a transcutaneous cannula with a plurality of radial openings.

There exists a need for improved apparatus and methods for inserting devices into a patient, such as devices for inserting transcutaneous cannulas for use with a patch pump.

### SUMMARY

Provided herein are devices, systems, and methods for inserting devices into a patient, such as devices for inserting transcutaneous cannulas for use with a patch pump. The implanted cannula may be used with a patch pump adhered to a wearer's body. An enhanced applicator needle is provided to cause the cannula transition from a straight configuration to a curved configuration beneath the outer skin layer during deployment. The applicator needle has a structural modification to cause the needle, and thereby cause the cannula having the needle positioned therein, to curve beneath the skin, for example, a special cut pattern at the needle tip and/or a bend at the distal region of the needle. Once transcutaneously implanted in the curved manner, the needle is fully withdrawn from the cannula so that a patch pump can be fluidly coupled to the curved cannula for transcutaneously delivering medication (e.g., insulin) to a target infusion area beneath the wearer's outer skin layer.

In accordance with some aspects, a device for inserting a cannula transcutaneously for use with a medication infusion device configured to be removably adhered to a wearer's skin is provided. The device may include an elongated shaft, a needle tip, and a structural modification. The elongated shaft defines a longitudinal axis and has a proximal region and a distal region. The elongated shaft may be configured to be disposed within a lumen in the cannula in a pre-deployment state. The needle tip is at the distal region of the elongated shaft and configured to pierce the wearer's skin. The structural modification may be at the distal region of the elongated shaft and configured to cause the elongated shaft and the cannula to curve within the wearer's skin in a deployment state for transcutaneously implanting the cannula.

The structural modification may include a bend in the elongated shaft at the distal region relative to the longitudinal axis. Additionally or alternatively, the structural modification includes a plurality of angled cuts at the needle tip. In some embodiments, a first angled cut of the plurality of angled cuts extends more proximally along the elongated shaft than other angled cuts of the plurality of angled cuts. The first angled cut's angle relative to the longitudinal axis may be less than the other angled cuts' angle relative to the longitudinal axis. The first angled cut may be positioned at the needle tip such that the elongated shaft and the cannula curve away from the first angled cut when deployed within the wearer's skin. The other angled cuts may consist of a second angled cut and a third angled cut.

The bend may be angled relative to the longitudinal axis so that the elongated shaft with the structural modification is configured to be fully withdrawn from the cannula via the lumen for transcutaneously implanting the cannula. The bend may be angled relative to the longitudinal axis such that the elongated shaft and the cannula curve towards the bend when deployed within the wearer's skin. In some embodiments, the bend is angled from 1 to 20 degrees from the longitudinal axis.

The elongated shaft may have a length longer than the cannula's length. There may be a proximal bend at the proximal region of the elongated shaft. The proximal bend may be orthogonal to the longitudinal axis. In some embodiments, the elongated shaft is straight between the proximal bend and the structural modification. The straight portion of the elongated shaft may be longer than the cannula's length. The proximal bend and the bend at the distal region may extend away from the longitudinal axis in the same direction.

The elongated shaft, the needle tip, the structural modification, and the proximal bend may be monolithic. The elongated shaft, the needle tip, the structural modification may be solid. The elongated shaft, the needle tip, the structural modification may be a metal needle.

The structural modification may be positioned on the elongated shaft distal to the cannula's distal end in the pre-deployment state. In some embodiments, the device is fully withdrawn from the cannula such that the cannula remains transcutaneously implanted within the wearer's skin. The cannula may remain curved within the wearer's skin after the device is fully withdrawn from the cannula. In some embodiments, the cannula's average angle relative to the wearer's skin surface at insertion is −10 to 45 degrees.

The cannula and the elongated shaft's section within the cannula may be straight in the pre-deployment state and configured to curve within the wearer's skin in the deployment state. The elongated shaft may be configured to curve in the deployment state, and thereby cause the cannula to curve in the deployment state, in a concave manner such that an open portion of the curve faces towards the wearer's skin surface. In some embodiments, the device further includes the cannula. The distal tip of the cannula may have an ogival shape.

In some embodiments, the elongated shaft, the needle tip, and the structural modification are a needle, and the device further includes an applicator configured to house the needle and the cannula in the pre-deployment state. The applicator may be configured to, upon actuation, cause the needle and the cannula to be inserted into the wearer's skin for transcutaneously implanting the cannula. The device may include an adhesive pad configured to be adhered to the wearer's skin. In the deployment state, the elongated shaft may be configured to be removed from the cannula while the cannula is coupled to the adhesive pad and remains transcutaneously implanted.

The device may include the medication infusion device. For example, the medication infusion device may be being a patch pump configured to be fluidly coupled to the transcutaneously implanted cannula after the elongated shaft, the needle tip, and the structural modification have been removed from the cannula via the lumen. The patch pump infuses medication to the wearer via the transcutaneously implanted cannula.

In accordance with another aspect, a method for making a device for inserting a cannula transcutaneously for use with a medication infusion device configured to be removably adhered to a wearer's skin is provided. The method may include providing an elongated shaft defining a longitudinal axis and having a proximal region and a distal region, the elongated shaft configured to be disposed within a lumen in the cannula in a pre-deployment state, the distal region of the elongated shaft having a needle tip configured to pierce the wearer's skin; and making a structural modification at the distal region of the elongated shaft so as to cause the elongated shaft and the cannula to curve within the wearer's skin in a deployment state for transcutaneously implanting the cannula.

Making the structural modification may include (i) making a bend in the elongated shaft at the distal region relative to the longitudinal axis; and/or (ii) making plurality of angled cuts at the needle tip, wherein a first angled cut of the plurality of angled cuts extends more proximally along the elongated shaft than other angled cuts of the plurality of angled cuts. Making the structural modification may occur before the elongated shaft is disposed within the lumen of the cannula. Making the structural modification may occur while the elongated shaft is disposed within the lumen of the cannula. After making the structural modification, the method may include moving the elongated shaft proximally relative to the cannula so as to position the structural modification closer to a distal end of the cannula.

### BRIEF DESCRIPTION OF THE DRAWINGS

These and other features, aspects, and advantages of the present invention will become apparent from the following description, appended claims, and the accompanying exemplary embodiments shown in the drawings, which are briefly described below.

FIG. 1 illustrates an exemplary medication infusion system having a patch pump for delivering medication in accordance with the principles of the present invention.

FIGS. 2A and 2B are, respectively, perspective and exploded views of an exemplary pad and applicator for attaching a pad and inserting a cannula in a curved manner.

FIG. 3A shows an exemplary needle for transcutaneously inserting the cannula for subsequent delivery of medication through the cannula.

FIG. 3B shows a close-up view of the distal region of the needle of FIG. 3A with a plurality of angled cuts at the needle tip for curved needle insertion.

FIG. 3C is a perspective view of the needle of FIG. 3A.

FIGS. 4A, 4B, and 4C are illustrations of various views of the distal region of another exemplary needle with a bend for causing curved insertion of the needle and cannula transcutaneously.

FIG. 5A is a cross-sectional side view of the applicator, pad, and cannula showing the needle and cannula inserted into the wearer in the curved manner.

FIG. 5B is a cross-sectional side view of the pad, cannula, and assembled pump showing how the microdosing tubing couples to the curved cannula to deliver microdoses of medication transcutaneously.

FIGS. 6A and 6B are perspective views of an exemplary cannula for delivering medication.

FIGS. 7A, 7B, and 7C show exemplary steps for making a needle device with a structural modification for curved transcutaneous insertion of the cannula.

FIGS. 8A and 8B show alternative exemplary steps for making a needle device with a structural modification for curved transcutaneous insertion of the cannula.

FIGS. 9A, 9B, and 9C are close-up cross-sectional and perspective views of the distal tip at the distal region of an exemplary cannula.

FIGS. 10A, 10B, and 10C are close-up cross-sectional and perspective views of the distal tip at the distal region of another exemplary cannula.

### DETAILED DESCRIPTION

Provided herein are systems and methods for implanting devices for delivering fluid to a patient. For example, medication such as insulin may be delivered transcutaneously using a cannula that is inserted into the patient's skin using an enhanced needle design. The transcutaneously implanted cannula may be coupled to a patch pump that is user-friendly, environmentally-friendly, lower cost, discreet, less prone to errors, and/or that provide precise, repeatable doses of medication.

The systems and methods described herein may be used to deliver medication including, but are not limited to, insulin, antibiotics, nutritional fluids, total parenteral nutrition or TPN, analgesics, morphine, hormones or hormonal drugs, gene therapy drugs, anticoagulants, analgesics, cardiovascular medications, AZT or chemotherapeutics. The types of medical conditions that the systems and methods might be used to treat include diabetes, cardiovascular disease, pain, chronic pain, cancer, ADDS, neurological diseases, Alzheimer's Disease, ALS, Hepatitis, Parkinson's Disease or spasticity. Preferably, the systems and methods are optimized for transcutaneous delivery of insulin to users with diabetes including Type I Diabetes Mellitus patients.

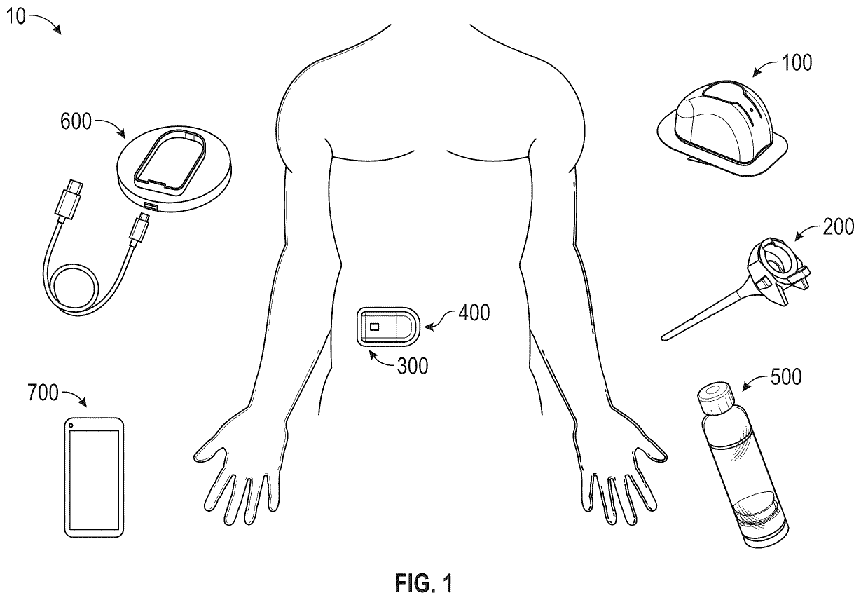

Referring to FIG. 1, an exemplary medication infusion system including a patch pump for delivering medication is described. In FIG. 1, components of the system are not depicted to scale on either a relative or absolute basis. Medication infusion system 10 may include applicator 100, cannula 200, pump 300, cap 400, cartridge 500, charging system 600, and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In a preferred embodiment, system 10 includes a second pump, such that the wearer may charge the second pump while using the first pump and vice versa. In this manner, the wearer will always have a pump that is charged and ready to be used once the cartridge of the pump in use is empty. Further, this system is designed to reduce waste while reducing the number of times the wearer is required to insert a new cannula. Medication infusion system 10 may be used to apply cannula 200 and a pad to a wearer and to deliver medication through cannula 200 via a patch pump coupled to the pad.

Applicator 100 is configured to apply an adhesive pad to the wearer and, upon actuation, to insert cannula 200 into the wearer using an improved needle design. The pad is configured to be secured to the wearer for a period of time, e.g., at least 3 days, 7-10 days, and then may be replaced by a similar pad using a similar applicator. The pad may include a pad skeleton having one or more locking mechanisms that are configured to couple the pad to applicator 100 for insertion of cannula 200 or to the assembled pump for delivery of medication. The portion of applicator 100 that inserts the needle, and thereby the cannula, is designed to be disposable after use. Applicator 100 may include an internal component configured to support an insertion mechanism designed to insert cannula 200 through the skin of the wearer via rotational movement and to guide and orient cannula 200 during insertion.

Applicator 100 may include an applicator needle configured to pierce the wearer's skin that is coupled to one or more links configured to interact with cannula 200. Upon actuation by the wearer, applicator 100 causes cannula 200 and the applicator needle within cannula 200 to be inserted through the wearer's skin. An enhanced applicator needle is provided to cause cannula 200 transition from a straight configuration to a curved configuration beneath the outer skin layer during deployment. The applicator needle has a structural modification to cause the needle, and thereby cause cannula 200 having the needle positioned therein, to curve beneath the skin. For example, as described in detail herein, the structural modification may be a special cut pattern at the needle tip and/or a bend at the distal region of the needle. Once transcutaneously implanted in the curved manner, the needle is fully withdrawn from cannula 200 so that a patch pump (e.g., pump 300 having cap 400 coupled thereto with cartridge 500 therein) can be fluidly coupled to the curved cannula 200 for transcutaneously delivering medication (e.g., insulin) to a target infusion area beneath the wearer's outer skin layer.

Cannula 200 may include a proximal cannula head configured to couple to one or more locking mechanisms on the pad skeleton and, at the same time, uncouple applicator 100 from the pad skeleton. The insertion mechanism further may be configured to continue rotating to withdraw the applicator needle from cannula 200 and to store the applicator needle within the applicator after cannula 200 is inserted.

Cannula 200 is designed to receive medication doses from a patch pump and to deliver the medication through one or more apertures. The one or more apertures may be disposed at the distal tip and/or along the elongated shaft of cannula 200 such that the medication is delivered along the length of elongated shaft. Preferably, the apertures are arranged and oriented such that the medication is delivered only below the derma layer of skin. The apertures may be oval in shape for, for example, better plastic injection process behavior. Cannula 200 may include a cannula head having a self-sealing septum configured to support and guide the applicator needle during insertion of cannula 200 and the outflow needle of cap 400 during delivery of medication. In some embodiments, cannula 200 may be designed to change the location at which medication is delivered to the patient via the aperture(s) over time without repositioning cannula 200 in the patient's skin, for example, using one or more biodegradable materials as described in U.S. Pat. No. 11,529,460, the entire contents of which are incorporated herein by reference.

Pump 300 is designed to pump medication from cartridge 500 through the microdosing system, through a transcutaneous portion, and into the wearer. The transcutaneous portion preferably includes cannula 200 inserted into the wearer's skin and having one or more apertures beneath the outer skin layer for delivery of the dose of medication. Pump 300 is designed to be removably coupled to cap 400 and the pad to form a patch pump, which is configured to deliver doses of medication through cannula 200 transcutaneously to the patient. The pump-cap assembly advantageously provides precise, repeatable microdoses of medication to the wearer. After a cartridge of medication is used, a battery within pump 300 is charged and, after charging, the cartridge and cap may be removed and discarded, leaving pump 300 ready to be used again with a new cartridge and a new cap. Pump 300 may include a motor disposed within the pump housing and may be configured to move a pusher towards a plunger of cartridge 500 such that insulin is advanced through an inflow needle of cap 400 and to a microdosing system designed to measure and deliver predetermined doses of medication.

Cap 400 preferably receives medication from cartridge 500 moved into tubing of cap 400 as a result of pumping by pump 300. Further, cap 400 may deliver predetermined doses of the medication through an outflow needle, into cannula 200, and to the wearer. Cap 400 preferably is designed to be replaced after the cartridge is empty or when the temperature sensor detects a temperature exceeding a predetermined temperature threshold, the temperature indicating that the insulin was damaged due to long exposure at a high temperature. Cap 400 may include a microdosing system configured to measure and deliver the predetermined doses of medication. Cap 400 further may include locking mechanisms configured to lock cap 400 to pump 300 and/or to the pad skeleton.

Cartridge 500 is an enclosed container designed to hold the medication for infusion into the patient. Cartridge 500 may be a commercially available insulin container such as the NovoRapid PumpCart available from Novo Nordisk A/S of Bagsværd, Denmark. Cartridge 500 preferably is pre-filled with a plurality of doses of medication such as insulin. The patch pump is designed such that when cartridge 500 is inserted into the pump patch, the cartridge 500 is completely encased by pump 300 and cap 400. Cartridge 500 may include a cartridge cap through which is disposed an inflow needle of cap 400. Cartridge 500 further may include a flexible plunger configured to be advanced towards the cartridge cap, responsive to pumping by pump 300. As the plunger is displaced, insulin is delivered to a microdosing system of cap 400, which in turn delivers predetermined doses of medication to the wearer one at a time. Once cartridge 500 is empty, it may be replaced by a similar pre-filled cartridge.

Charging system 600 is configured to charge one or more batteries within pump 300, e.g., via respective inductive coils disposed within the housing of a charger and pump 300. The charger is delivered with a USB-C to USB-A cable. The cable may be plugged into a standard USB-A socket (e.g. on an adapter put into a conventional wall electrical socket, on a computer, or in public transport), for charging components within the charger to permit charging pump 300.

Software application 700 is designed to cause a computer (e.g., smartphone, laptop, desktop, tablet, smartwatch, etc.) to communicate data with pump 300 and display information on the pump to a wearer in a user-friendly manner. Software application 700 may cause the computer to securely exchange data between two or more pumps that are used by a single wearer. Further, as described above, software application 700 may receive information from the continuous glucose monitoring sensor or other monitoring systems, for example, sensed glucose levels, information about patient food intake, and/or information about patient's activity levels (e.g., due to exercising, playing sports). In this manner, the pump is modular and interchangeable with many continuous glucose monitoring sensors or other monitoring systems, making the pump “universal.” Advantageously, the patch pump described herein may be used with a minimal amount of external monitoring while still being effective at delivering accurate microdoses of medication at levels to treat the wearer.

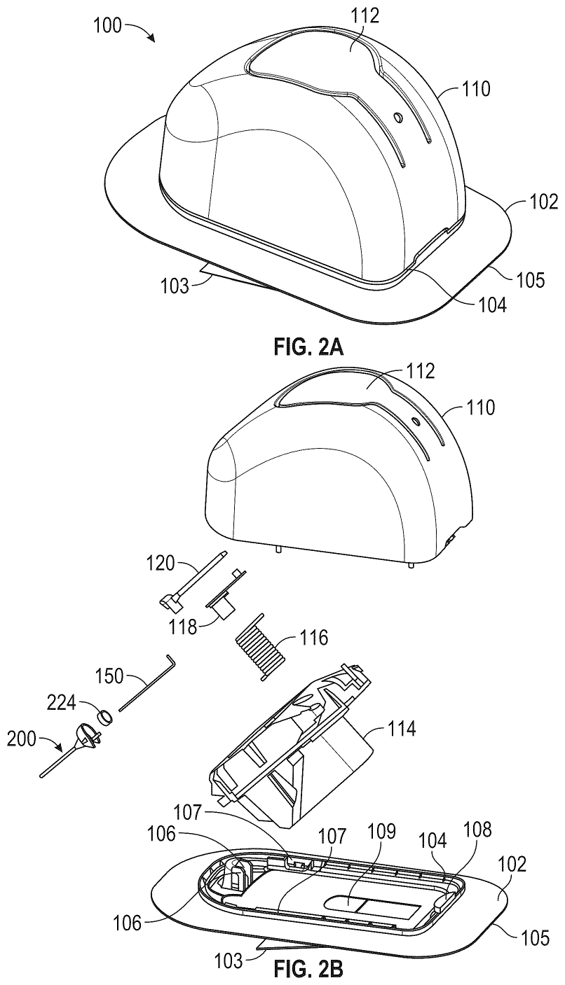

Referring now to FIGS. 2A and 2B, perspective and exploded views of an exemplary pad and applicator are described. Applicator 100 may transcutaneously apply a cannula, upon actuation by a user, which is designed to deliver doses of medication (e.g., insulin) from a patch pump configured to be removably coupled to the cannula. Advantageously, applicator 100 further may apply a pad that is adhered to the wearer's skin and then coupled to the patch pump. For example, actuation of applicator 100 may both insert the cannula and cause the cannula to be locked to the adhesive pad in a single actuation. Further, applicator 100 may include internal components designed to minimize noise during the actuation process. For example, applicator 100 may avoid clicks and/or hard stops that make audible noises during insertion of the cannula.

In a pre-actuation state, applicator 100 may be coupled to pad 102 as shown in FIG. 2A. For example, applicator 100 may be coupled to pad 102 via pad skeleton 104 of pad, which is disposed on a first surface of pad 102. Skin-safe pad adhesive 105 may be disposed on a second, skin-facing surface of pad 102 such that the pump-pad assembly may be attached to a wearer for a period of time, for example, 3-5 days, 3-10 days, or 10 days or more. One or more release liners 103 may be attached to pad adhesive 105 until pad 102 is ready to be secured to the wearer. Pad skeleton 104 may be a frame with a shape designed to surround the pump-cap assembly so as to securely couple the adhesive pad to the pump-cap for wearing by the patient. Pad skeleton 104 may be designed to removably couple portions of pad 102 to applicator 100 in the pre-actuation state. For example, pad skeleton 104 may have one or more attachment mechanisms to lock pad 102 to applicator 100 and unlock upon actuation of applicator 100. Advantageously, the attachment mechanisms also may lock the cannula to pad 102 after actuation. As depicted in FIG. 2A, pad skeleton 104 may have pad attachments 106 at a first end of pad 102 and pad back clip 108 at a second end of pad 102. Pad attachments 106 and pad back clip 108 may interact with applicator 100 or a patch pump to lock the pad to applicator 100 or the patch pump. Pad attachments 106 may include at least two arms that protrude upwards from the pad and away from the skin surface of the wearer. Each arm may have an opening (e.g., slot) to receive extensions from the applicator during pre-actuation and extensions from the cannula post-actuation. Thus, the arms, which may have a U-shape, and openings may be used to lock to both the applicator and the cannula. Pad skeleton 104 may also include pad clips holes 107 disposed on the sides of pad skeleton 104. Pad clips holes 107 may be a hole or receptacle sized and shaped to interact with a corresponding feature of the pump-cap assembly such that the pump-cap assembly may be locked to the pad. Further, pad 102 may include pad opening 109 to allow direct sensing of the wearer's skin by one or more sensors of the pump. For example, the skin sensor(s) and/or the PPG sensor(s) may be positioned at pad opening 109 when the pump is coupled to the pad.

Applicator 100 may include applicator housing 110 and actuator 112. Applicator housing 110 is configured to house the mechanisms for inserting the cannula. After insertion of the cannula, internal component 114 is designed to withdraw and safely store needle 150 used to pierce the wearer's skin. Actuator 112, upon actuation, causes the cannula to be transcutaneously inserted into the wearer's skin. Actuation of actuator 112 also may unlock applicator 100 from pad 102. Actuation of actuator 112 also may lock the transcutaneously inserted cannula into pad 102. For example, actuation of applicator 100 may insert the cannula transcutaneously, unlock the applicator from the pad, and lock the cannula to the pad in a single actuation. Actuator 112 may release the internal mechanism disposed within applicator housing 110 when actuated by the wearer, thus causing the cannula to advance through the wearer's skin. Actuator 112 may be a button configured to be pressed by the wearer as illustrated, or may be a lever, snap, knob, or the like. The mechanism for inserting the cannula may include internal component 114, biasing member 116, links 118 and 120, and needle 150 which are disposed within applicator housing 110, and are configured to advance cannula 200 through pad 102 and into the wearer's skin. Needle 150 is configured to be disposed within cannula 200 during insertion and to be withdrawn from cannula 200 after insertion. Self-sealing septum 224 may be disposed within the cannula head of cannula 200 in order to support and guide needle 150 and minimize backflow out of cannula 200.

Needle 150 is designed to cause cannula 200 transition from a straight configuration to a curved configuration beneath the outer skin layer during deployment. Needle 150 preferably has a structural modification to cause needle 150, and thereby cause cannula 200 having needle 150 positioned therein, to curve beneath the skin. For example, as described below, the structural modification may be a special cut pattern at the needle tip and/or a bend at the distal region of needle 150.

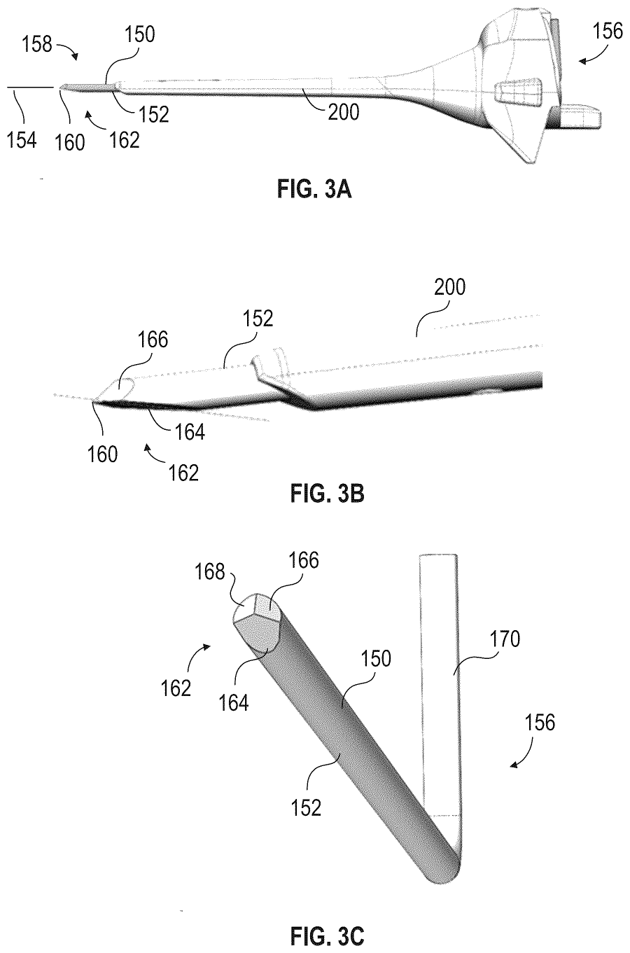

FIG. 3A shows exemplary needle 150 for transcutaneously inserting cannula 200 for subsequent delivery of medication through cannula 200. For example, needle 150 may be used to insert cannula 200 transcutaneously for use with a medication infusion device (e.g., a patch pump (e.g., pump 300 having cap 400 coupled thereto with cartridge 500 therein)) configured to be removably adhered to a wearer's skin. Needle 150 may include elongated shaft 152 defining longitudinal axis 154 and having proximal region 156 and distal region 158. Elongated shaft 152 is sized and shaped to be disposed within a lumen in cannula 200 in a pre-deployment state, as shown in FIG. 3A. Needle 150 has needle tip 160 at distal region 158 of elongated shaft 152 configured to pierce the wearer's skin. Needle 150 has structural modification 162 at distal region 158 of elongated shaft 152 to cause elongated shaft 152 and cannula 200 to curve within the wearer's skin in a deployment state for transcutaneously implanting cannula 200.

Structural modification 162 may include a plurality of angled cuts at needle tip 160 and/or a bend in elongated shaft 152 at distal region 158 relative to longitudinal axis 154, as described in detail below.

FIG. 3B shows a close-up view of distal region 158 with a plurality of angled cuts at needle tip 160. First angled cut 164 of the plurality of angled cuts extends more proximally along elongated shaft 152 than other angled cuts of the plurality of angled cuts. Arranging the angled cuts in this manner causes elongated shaft 152, and thereby cannula 200, to bend and curve during deployment beneath the wearer's skin. The angle of first angled cut 164 relative to longitudinal axis 154 may be less than the other angled cuts' angle relative to longitudinal axis 154 as shown. First angled cut 164 is positioned at needle tip 160 such that elongated shaft 152 and cannula 200 curve away from first angled cut 164 when deployed within the wearer's skin.

FIG. 3C is a perspective view of needle 150. As shown, the plurality of angled cuts is three angled cuts: first angled cut 164, second angled cut 166, and third angled cut 168. As shown in FIG. 3A, elongated shaft 152 has a length longer than the length of cannula 200. As best shown in FIG. 3C, needle 150 may further include proximal bend 170 at proximal region 156 of elongated shaft 152. Proximal bend 170 may be used to couple needle 150 to internal components within applicator 100 so that applicator can insert cannula 200 transcutaneously upon actuation. As illustrated, proximal bend 170 may be orthogonal to longitudinal axis 154. Elongated shaft 152 may be straight between proximal bend 170 and structural modification 162. As shown in FIG. 3A, the straight portion of elongated shaft 152 is longer than the cannula's length.

Referring back to FIG. 3C, needle 150 is preferably one component. For example, elongated shaft 152, needle tip 160, and structural modification 162 are monolithic. Proximal bend 170 also may be monolithic with those other sections. Elongated shaft 152, needle tip 160, and structural modification 162 are preferably solid (without a lumen(s)). For example, elongated shaft 152, needle tip 160, and structural modification 162 are a metal needle.

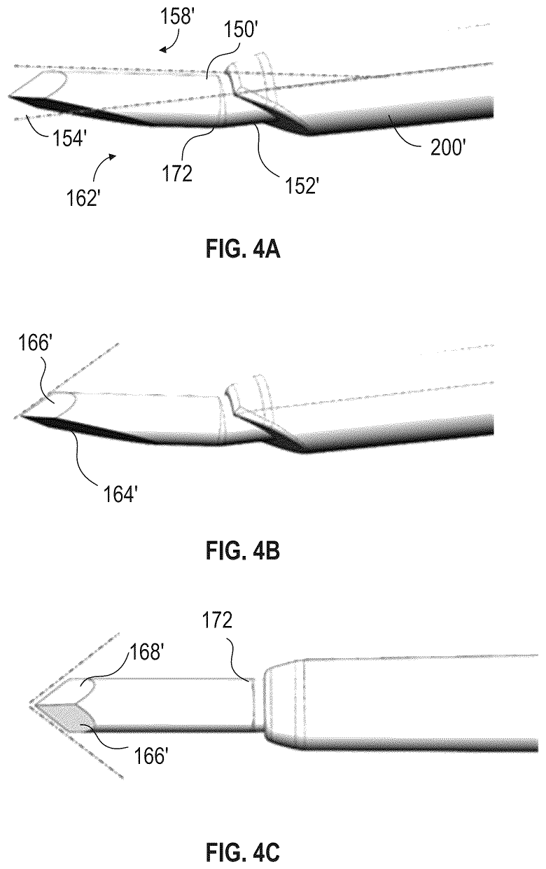

Referring now to FIGS. 4A, 4B, and 4C, exemplary needle 150′ is constructed similarly to needle 150 described above, wherein like components are identified by like-primed reference numbers. Thus, for example, elongated shaft 152′ in FIG. 4A corresponds to elongated shaft 152 of FIG. 3A, etc. except that structural modification 162′ is different than structural modification 162. As shown, structural modification 162′ further includes bend 172 in elongated shaft 152′ at the distal region 158′ relative to longitudinal axis 154′. Structural modification 162′ also includes the cutting angles of structural modification 162 although needle 150′ is not limited thereto.

As shown in FIG. 4A, bend 172 is angled relative to longitudinal axis 154′ so that elongated shaft 152′ with structural modification 162′ is configured to be fully withdrawn from cannula 200′ via the lumen for transcutaneously implanting cannula 200′. Bend 172 is angled in a limited manner so that needle 150′ is retractable through the lumen of cannula 200′ and its septum. For example, bend 172 may be angled from 1 to 20 degrees from longitudinal axis 154′.

FIGS. 4B and 4C show an exemplary cut pattern at the needle tip similar to the cut pattern in FIGS. 3A-3C above with first angled cut 164′, second angled cut 166′, and third angled cut 168′.

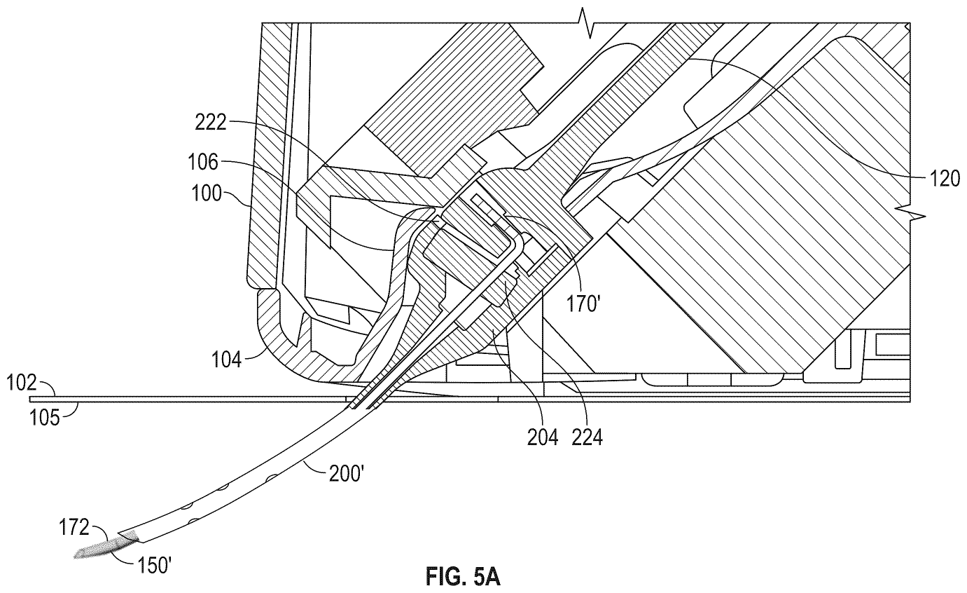

Referring now to FIGS. 5A and 5B, cross-sectional views showing applicator 100 applying needle 150′ and cannula 200′ transcutaneously in the deployment state in the curved manner (FIG. 5A) and the patch pump coupled to curved cannula 200′ after the needle 150′ and applicator 100 have been withdrawn and removed (FIG. 5B) for transcutaneously delivering medication via the patch pump through curved cannula 200′.

As shown in FIG. 5A, bend 172 is angled relative to the longitudinal axis such that the elongated shaft and cannula 200′ curve towards bend 172 when deployed within the wearer's skin. In some embodiments, the elongated shaft is configured to curve in the deployment state, and thereby cause cannula 200′ to curve in the deployment state, in a concave manner as shown in FIG. 5A such that an open portion of the curve faces towards the wearer's skin surface. In this manner, the depth relative to skin surface of curved penetration is reduced for a given cannula length compared to straight penetration.

Proximal bend 170′ and bend 172 at the distal region may extend away from the longitudinal axis in the same direction, as illustrated. Structural modification 162′ may be positioned on the elongated shaft distal to the cannula's distal end in the pre-deployment state and further may remain distal to the cannula's distal end until needle 150′ is withdrawn from cannula 200′ while cannula 200′ remains transcutaneously implanted in the wearer. Cannula 200′ and the elongated shaft's section within cannula 200′ are straight in the pre-deployment state (see, for example, FIGS. 3A, 7C, and 8B) and are configured to curve within the wearer's skin in the deployment state as shown in FIG. 5A. The elongated shaft's and the cannula's average angle relative to the wearer's skin surface at insertion is preferably in a range of −10 to 45 degrees. When the needle is withdrawn from the cannula, the cannula's average angle relative to the wearer's skin surface at insertion remains at −10 to 45 degrees.

In FIG. 5A, the applicator is depicted in a partially-deployed state, wherein the cannula is inserted into the skin of the wearer, but needle 150′ is not yet withdrawn. The distal end of needle 150′ may be disposed distal to the cannula tip and the proximal end of needle 150′ may be coupled to link 120. Needle 150′ preferably is inserted through the septum of the cannula and extends past the distal end of the cannula. Septum 224 is a self-sealing material designed to seal the proximal region of the cannula, such as silicone, and minimizes backflow out of the cannula. Septum 224 may be disposed within cannula head 204 and supports and guides needle 150′ such that the needle is withdrawn in a substantially linear direction. Needle 150′ may be coupled to link 120, which interacts with applicator interfaces 220 and 222 disposed on cannula head 204 and configured to provide smooth and continuous contact with link 120 during insertion of the cannula. After the cannula is inserted, cannula head 204 of the cannula may be locked to pad attachments 106. The elongated shaft of the cannula extends from pad attachments 106, past pad skeleton 104, through pad 102, and into the skin of the wearer. Depending on the type of medication inserted (e.g., insulin), the cannula may be inserted such that apertures of the cannula and the cannula tip are disposed below the dermal layer of skin.

Applicator 100 houses the needle and the cannula in the pre-deployment state. Applicator 100 is configured to, upon actuation, cause the needle and the cannula to be inserted into the wearer's skin for transcutaneously implanting the cannula. Adhesive pad 102 is configured to be adhered to the wearer's skin. In the deployment state, the elongated shaft is configured to be removed from the cannula while the cannula is coupled to the adhesive pad and remains transcutaneously implanted.

FIG. 5B shows the patch pump coupled to curved cannula 200′ after needle 150′ and applicator 100 have been withdrawn and removed for transcutaneously delivering medication via the patch pump through curved cannula 200′. After the cannula is deployed and is coupled to pad 102 via pad attachments 106, the applicator is removed from pad 102. A patch pump, which preferably includes a disposable cap and a reusable pump, is coupled to pad 102 to deliver medication via the cannula. For example, the patch pump may include a housing that is configured to lock to pad skeleton 104 such that the patch pump is secured to the wearer during the wearer's normal, daily motions. The patch pump may include outflow needle 408, which is in fluid communication with the cannula and to deliver a predetermined dose of medication from a cartridge to the cannula responsive to pumping at the patch pump. Outflow needle 408 preferably is configured to pierce septum 224 of cannula head 204 such that the distal end of outflow needle 408 is disposed below septum 224 and medication flows into the elongated shaft of the cannula, rather than back into the patch pump, for transcutaneous delivery to the wearer via one or more apertures of the cannula. Further, as illustrated, the entirety of cannula head 204 may be designed to stay above the skin line and remain external to the patient while elongated shaft of the cannula is transcutaneous.

As explained above, the needle is fully withdrawn from the cannula such that the cannula remains transcutaneously implanted within the wearer's skin. As shown in FIG. 5B, cannula 200′ remains curved within the wearer's skin after the needle is fully withdrawn from the cannula.

In the examples when the medication infusion device is a patch pump configured to be fluidly coupled to the transcutaneously implanted cannula after the needle (elongated shaft, the needle tip, and the structural modification) has been removed from the cannula via the cannula's lumen, the patch pump can then infuse medication to the wearer via the transcutaneously implanted cannula.

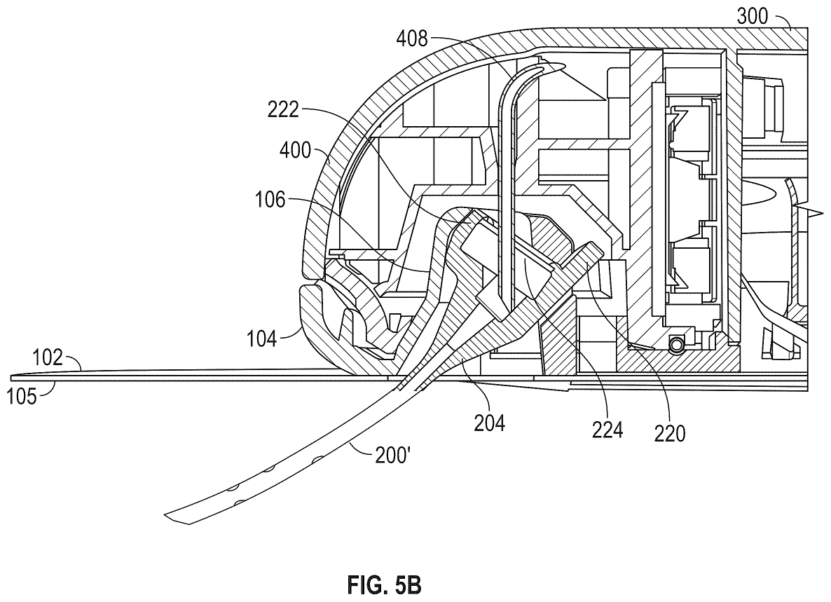

Referring now to FIGS. 6A and 6B, an exemplary cannula for delivering medication is described. Cannula 200 may be injection molded from a single piece of material, which is preferable to extrusion in order to reduce the risk of kinking of cannula 200. Cannula 200 preferably is made from a material that is insulin compatible and flexible and includes cannula head 204, cannula tip 218, and elongated shaft 202. Elongated shaft 202 is designed to be straight prior to deployment (FIG. 6A) and to curve, responsive to curving of the needle disposed therein, when inserted in the skin (FIG. 6B). The cross-section of cannula 200 may have an oval section shape along the extended shaft for better curve. In this manner, the cannula is thinner in the direction of the curve. Cannula head 204 is disposed at the proximal end of cannula 200 and configured to interact with the applicator needle and the needle through which the medication is delivered. Cannula tip 218 is disposed at the distal end of cannula 200 and may include distal aperture 216 for delivering medication. Elongated shaft 202 may extend between cannula head 204 and cannula tip 218 and may include one or more apertures for delivery of medication. Elongated shaft 202 may increase in diameter towards cannula head 204 and the wearer's skin surface, to mitigate the risk that the delivered medication travels proximally along the outside surface of the cannula to the dermal layer or the surface of the skin. This conical shape may also reduce the risk of kinking of cannula 200.

Elongated shaft 202 also may have one or more apertures disposed along the elongated shaft in any configuration. Preferably the apertures are disposed such that medication is delivered only below the dermal layer of the skin. As depicted in FIG. 6A, cannula 200 may include aperture 210 and aperture 214, disposed distal to aperture 210, which are oriented towards the skin surface of the wearer. As shown in FIG. 6B, cannula 200 also may include aperture 208 and aperture 212, which are oriented away from the skin surface of the wearer. As will also be understood by one of ordinary skill in the art, the cannula may be configured such that the apertures are axially oriented relative to a different target infusion area within the wearer.

Cannula head 204 may include one or more applicator interfaces that are configured to interact with link 120 to permit rotational movement of the cannula during insertion of the cannula into the skin of the wearer. For example, applicator interface 220 may be disposed on the side of cannula head 204 that is farthest away from the skin surface of the wearer. Applicator interface 220 may be a rounded, convex protrusion, which interacts with a corresponding rounded, concave receptacle of link 120. Cannula head 204 also may include applicator interface 222, which may be disposed on the opposite side of the cannula head, the side closest to the skin surface of the wearer. Applicator interface 222 may be a rounded, concave receptacle, which interacts with a corresponding rounded, convex protrusion of link 120. The rounded shapes of applicator interfaces 220 and 222 and the corresponding features of link 120 are designed such that link 120 maintains smooth and continuous contact with cannula head 204 during insertion of cannula 200 into the wearer's skin.

Cannula head 204 also may include one or more clips 206 configured to guide cannula 200 in a substantially linear direction. Clips 206 may be any component of cannula head 204 that is configured to interact with the channel of the internal component of the applicator during insertion of cannula 200 into the wearer's skin. For example, clips 206 may be one or more wings disposed on a first and second side of cannula head 204 and sized and shaped to slide along the ledges of the channel. Clips 206 alternatively may be receptacles disposed on cannula head 204 and configured to slide along corresponding protrusions of the channel. Cannula head 204 may further include wings 207, which may be configured to interact with the guiding arm to order to prevent cannula 200 from rotating around the longitudinal axis of cannula 200 during and after insertion. Preferably, wings 207 are configured to protrude towards the wearer's skin and the guiding arm and are sized and shaped such that the guiding arm fits between the two wings. Clips 206 and wings 207 are designed to control orientation of the cannula during delivery and insertion. Because the apertures along the shaft of the cannula may be radially and longitudinally offset from one another, control of the orientation of the cannula in the wearer's skin is important to ensure precise delivery of medication through the aperture(s). Thus, clips 206 and wings 207 ensure axial orientation in a target direction of the apertures.

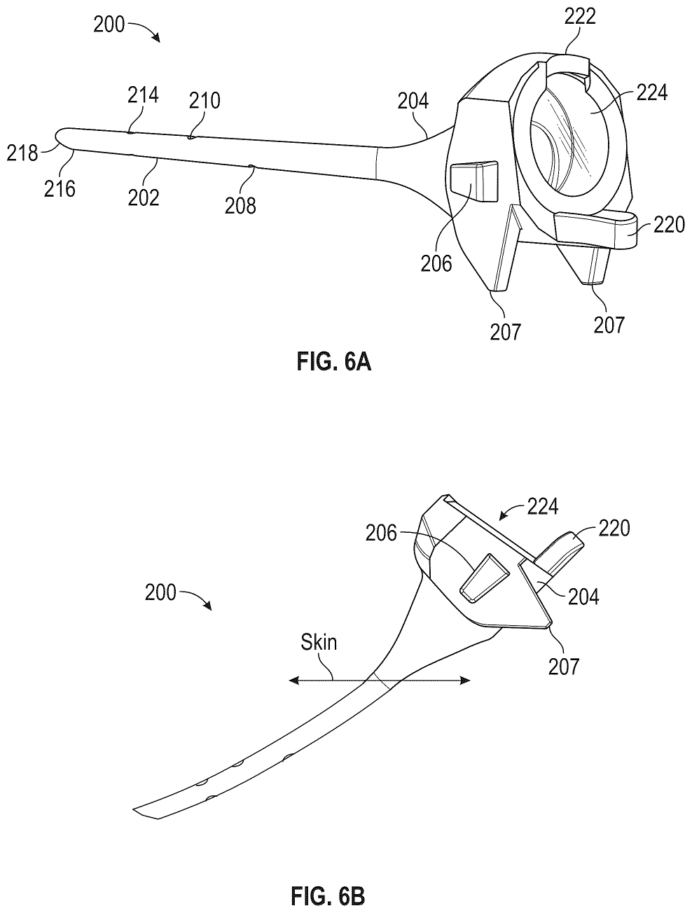

Referring now to FIGS. 7A, 7B, and 7C, exemplary steps for making a needle device with a structural modification for curved transcutaneous insertion of the cannula are shown. The method may be used for making a device for inserting cannula 200′ transcutaneously for use with a medication infusion device configured to be removably adhered to a wearer's skin. Needle 150′ with an elongated shaft defining a longitudinal axis and having a proximal region and a distal region is provided. The elongated shaft of the needle is configured to be disposed within a lumen in cannula 200′ in a pre-deployment state. The distal region of the elongated shaft has a needle tip configured to pierce the wearer's skin, as described above.

A structural modification is made at the distal region of the elongated shaft so as to cause the elongated shaft and the cannula to curve within the wearer's skin in a deployment state for transcutaneously implanting the cannula. Making the structural modification may include making bend 172 in the elongated shaft at the distal region relative to the longitudinal axis as shown in FIG. 7B and/or (ii) making plurality of angled cuts at the needle tip. As explained above, a first angled cut of the plurality of angled cuts may extend more proximally along the elongated shaft than other angled cuts of the plurality of angled cuts.

As shown in FIG. 7B, the structural modification may be made while elongated shaft 152′ is disposed within the lumen of cannula 200′. For example, bend 172 may be formed after needle 150′ is slid in cannula 200′ while pushing needle 150′ as far as possible distally (FIG. 7A), bending (FIG. 7B), and then shifting needle 150′ backward to the position for deployment (FIG. 7C). In this manner, after making the structural modification, the elongated shaft may be moved proximally relative to the cannula so as to position the structural modification closer to a distal end of the cannula.

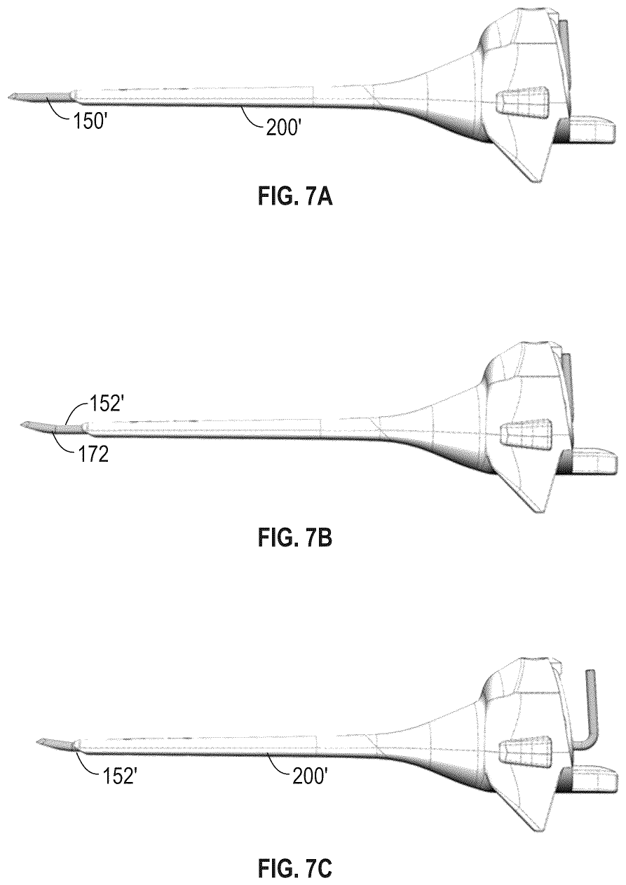

FIGS. 8A and 8B show alternative exemplary steps for making a needle device with a structural modification for curved transcutaneous insertion of the cannula. As shown in FIG. 8A, the structural modification is made before the elongated shaft is disposed within the lumen of the cannula. For example, elongated shaft 152′ is bent at the distal region of needle 150′ to form bend 172 before insertion into cannula 200′.

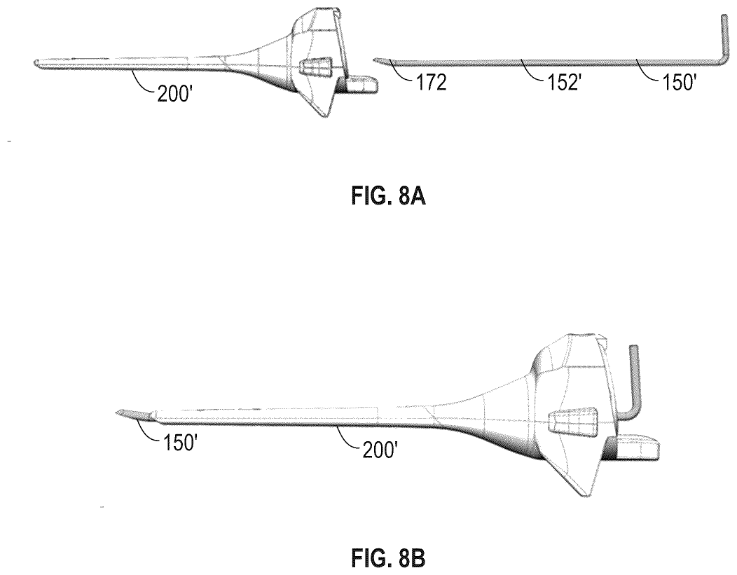

FIGS. 9A, 9B, and 9C are close-up views of the distal tip at the distal region of cannula 200, where FIGS. 9A and 9C are cross-sectional views and FIG. 9B is a perspective view. As shown, cannula tip 218 is disposed at the distal end of cannula 200 and includes distal aperture 216 for delivering medication via lumen 226 that extends through cannula 200. As shown, a portion (e.g., half) of cannula tip 218 is angled to facilitate cannula insertion.

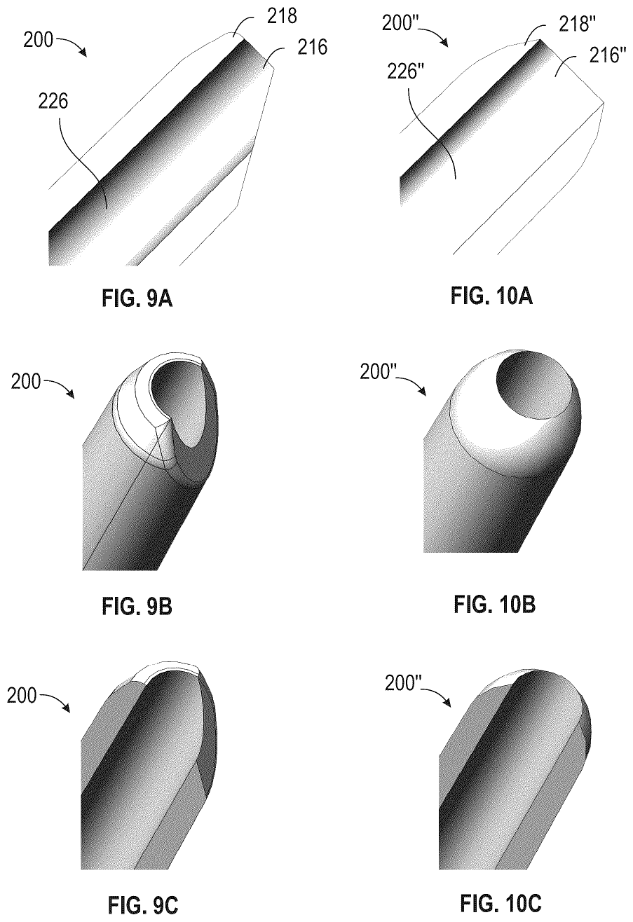

FIGS. 10A, 10B, and 10C are close-up views of the distal tip at the distal region of cannula 200″, where FIGS. 10A and 10C are cross-sectional views and FIG. 10B is a perspective view. Cannula 200″ is constructed similarly to cannula 200 described above, wherein like components are identified by like-primed reference numbers. Thus, for example, lumen 226 in FIG. 9A corresponds to lumen 226″ of FIG. 10A, etc. except that cannula tip 218″ is different than cannula tip 218. As shown, cannula tip 218″ of FIGS. 10A, 10B, and 10C has an ogival shape extending around the circumference at the tip.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed is:

1. A device for inserting a cannula transcutaneously for use with a medication infusion device configured to be removably adhered to a wearer's skin, the device comprising:
an elongated shaft defining a longitudinal axis and having a proximal region and a distal region, the elongated shaft configured to be disposed within a lumen in the cannula in a pre-deployment state;
a needle tip at the distal region of the elongated shaft configured to pierce the wearer's skin; and
a structural modification at the distal region of the elongated shaft configured to cause the elongated shaft and the cannula to curve within the wearer's skin in a deployment state for transcutaneously implanting the cannula.

2. The device of claim 1, wherein the structural modification comprises a bend in the elongated shaft at the distal region relative to the longitudinal axis.

3. The device of claim 1, wherein the structural modification comprises a plurality of angled cuts at the needle tip.

4. The device of claim 3, wherein a first angled cut of the plurality of angled cuts extends more proximally along the elongated shaft than other angled cuts of the plurality of angled cuts.

5. The device of claim 4, wherein the first angled cut's angle relative to the longitudinal axis is less than the other angled cuts' angle relative to the longitudinal axis.

6. The device of claim 4, wherein the first angled cut is positioned at the needle tip such that the elongated shaft and the cannula curve away from the first angled cut when deployed within the wearer's skin.

7. The device of claim 4, wherein the other angled cuts consist of a second angled cut and a third angled cut.

8. The device of claim 2, wherein the bend is angled relative to the longitudinal axis so that the elongated shaft with the structural modification is configured to be fully withdrawn from the cannula via the lumen for transcutaneously implanting the cannula.

9. The device of claim 2, wherein the bend is angled relative to the longitudinal axis such that the elongated shaft and the cannula curve towards the bend when deployed within the wearer's skin.

10. The device of claim 2, wherein the bend is angled from 1 to 20 degrees from the longitudinal axis.

11. The device of claim 1, wherein the elongated shaft has a length longer than the cannula's length.

12. The device of claim 1, further comprising a proximal bend at the proximal region of the elongated shaft.

13. The device of claim 12, wherein the proximal bend is orthogonal to the longitudinal axis.

14. The device of claim 12, wherein the elongated shaft is straight between the proximal bend and the structural modification.

15. The device of claim 14, wherein the straight portion of the elongated shaft is longer than the cannula's length.

16. The device of claim 12, wherein the proximal bend and the bend at the distal region extend away from the longitudinal axis in the same direction.

17. The device of claim 12, wherein the elongated shaft, the needle tip, the structural modification, and the proximal bend are monolithic.

18. The device of claim 1, wherein the elongated shaft, the needle tip, the structural modification are solid.

19. The device of claim 1, wherein the elongated shaft, the needle tip, the structural modification are a metal needle.

20. The device of claim 1, wherein the structural modification is positioned on the elongated shaft distal to the cannula's distal end in the pre-deployment state.

21. The device of claim 1, wherein the device is fully withdrawn from the cannula such that the cannula remains transcutaneously implanted within the wearer's skin.

22. The device of claim 21, wherein the cannula remains curved within the wearer's skin after the device is fully withdrawn from the cannula.

23. The device of claim 22, wherein the cannula's average angle relative to the wearer's skin surface at insertion is −10 to 45 degrees.

24. The device of claim 1, wherein the cannula and the elongated shaft's section within the cannula are straight in the pre-deployment state and are configured to curve within the wearer's skin in the deployment state.

25. The device of claim 1, wherein the elongated shaft is configured to curve in the deployment state, and thereby cause the cannula to curve in the deployment state, in a concave manner such that an open portion of the curve faces towards the wearer's skin surface.

26. The device of claim 1, further comprising the cannula.

27. The device of claim 26, wherein a distal tip of the cannula has an ogival shape.

28. The device of claim 1, wherein the elongated shaft, the needle tip, and the structural modification are a needle, the device further comprising an applicator configured to house the needle and the cannula in the pre-deployment state, and
wherein the applicator is configured to, upon actuation, cause the needle and the cannula to be inserted into the wearer's skin for transcutaneously implanting the cannula.

29. The device of claim 1, further comprising an adhesive pad configured to be adhered to the wearer's skin,
wherein, in the deployment state, the elongated shaft is configured to be removed from the cannula while the cannula is coupled to the adhesive pad and remains transcutaneously implanted.

30. The device of claim 1, further comprising the medication infusion device, the medication infusion device being a patch pump configured to be fluidly coupled to the transcutaneously implanted cannula after the elongated shaft, the needle tip, and the structural modification have been removed from the cannula via the lumen, the patch pump configured to infuse medication to the wearer via the transcutaneously implanted cannula.

31. A method for making a device for inserting a cannula transcutaneously for use with a medication infusion device configured to be removably adhered to a wearer's skin, the method comprising:
providing an elongated shaft defining a longitudinal axis and having a proximal region and a distal region, the elongated shaft configured to be disposed within a lumen in the cannula in a pre-deployment state, the distal region of the elongated shaft having a needle tip configured to pierce the wearer's skin; and
making a structural modification at the distal region of the elongated shaft so as to cause the elongated shaft and the cannula to curve within the wearer's skin in a deployment state for transcutaneously implanting the cannula.
